(19) United States
(12) Patent Application Publication
(10) Pub. No.: US 2019/0043215 A1
(43) Pub. Date: Feb. 7, 2019

(54) ENDOSCOPE APPARATUS

(71) Applicant: OLYMPUS CORPORATION, Tokyo (JP)

(72) Inventors: Seigo ITO, Tokyo (JP); Shunya AKIMOTO, Kawasaki-shi (JP); Jun HASEGAWA, Tokyo (JP); Junichi ONISHI, Tokyo (JP)

(73) Assignee: OLYMPUS CORPORATION, Tokyo (JP)

(21) Appl. No.: 16/156,076

(22) Filed: Oct. 10, 2018

Related U.S. Application Data
(63) Continuation of application No. PCT/JP2017/011397, filed on Mar. 22, 2017.

(30) Foreign Application Priority Data
May 25, 2016 (JP) 2016-104525

(51) Int. Cl. G06T 7/70; A61B 1/00; G06T 7/55; G06T 17/00; G06T 19/20; G06T 7/194; A61B 34/20; A61B 90/00

(52) U.S. Cl. CPC G06T 7/70; A61B 2034/105; G06T 7/55; G06T 17/00; G06T 19/20; G06T 7/194; A61B 34/20; A61B 90/37; G06T 2207/10028; G06T 2207/10068; G06T 2200/08; G06T 2207/30084; G06T 2219/004; G06T 2219/2004; A61B 1/00009; A61B 1/00193

(57) ABSTRACT

An endoscope apparatus includes: an endoscope configured to acquire an image of an inside of a subject; and a processor including hardware. The processor generates three-dimensional model data of the subject; generates a three-dimensional model image visually confirmable in a predetermined line-of-sight direction, based on the three-dimensional model data; generates progress information enabling a progress state of endoscopic observation to be visually confirmed as a ratio; and associates the progress information with the three-dimensional model image and presents the progress information relative to the three-dimensional model image side by side.

### CROSS REFERENCE TO RELATED APPLICATION

This application is a continuation application of PCT/JP2017/011397 filed on Mar. 22, 2017 and claims benefit of Japanese Application No. 2016-104525 filed in Japan on May 25, 2016, the entire contents of which are incorporated herein by this reference.

### BACKGROUND OF THE INVENTION

### 1. Field of the Invention

The present invention relates to an endoscope apparatus that generates and enables display of a three-dimensional model image of a subject at the time of performing endoscopic observation.

### 2. Description of the Related Art

Endoscopic observation support techniques of generating a three-dimensional model image of a luminal organ and presenting an unobserved area to a surgeon on the generated three-dimensional model image are known.

For example, in International Publication No. 2012/101888, a medical apparatus is described which generates an insertion route through which a distal end portion of an insertion portion is to be inserted as far as a target site, based on a three-dimensional image data of a subject acquired in advance, and displays the generated insertion route being superimposed on a tomographic image generated from three-dimensional image data. In the patent publication, it is further described that an insertion route which has already been passed through and an insertion route as far as the target position are displayed on the three-dimensional model image with different line types.

In Japanese Patent Application Laid-Open Publication No. 2016-002206, a medical information processing system is described in which an observation image of a subject and information about an observation site included in past examination information about the subject are displayed on a display device, and site observation completion information showing that observation of the observation site corresponding to the information displayed on the display device has been completed is registered. Furthermore, in the patent publication, a technique of displaying sites for which observation has been completed, a site to be observed next and unobserved sites are displayed, for example, by square marks, a triangle mark and circle marks, respectively.

By using such endoscopic observation support techniques, it is possible to visually determine approximate positions of and an approximate number of unobserved areas, which is useful for preventing oversight.

### SUMMARY OF THE INVENTION

An endoscope apparatus according to one aspect of the present invention includes: an endoscope configured to acquire an image of an inside of a subject; and a processor including hardware; wherein the processor generates three-dimensional model data of the subject; generates a three-dimensional model image visually confirmable in a predetermined line-of-sight direction, based on the generated three-dimensional model data; generates progress information enabling a progress state of observation by the endoscope to be visually confirmed as a ratio on an observation target based on the three-dimensional model data; and associates the progress information with the three-dimensional model image and presents the progress information relative to the three-dimensional model image side by side.

### BRIEF DESCRIPTION OF THE DRAWINGS

FIG. 1 is a block diagram showing a configuration of an endoscope apparatus of a first embodiment of the present invention;

FIG. 2 is a diagram showing a state of a display screen of a display device including a progress information display portion of a first example, during observation, in the above first embodiment;

FIG. 3 is a diagram showing a state of the progress information display portion of the first example at the time of starting observation in the above first embodiment;

FIG. 4 is a diagram showing a state of the progress information display portion of a second example at the time of starting observation in the above first embodiment;

FIG. 5 is a diagram showing a state of the progress information display portion of the second example during the observation in the above first embodiment;

FIG. 6 is a flowchart showing operation of the endoscope apparatus of the above first embodiment;

FIG. 7 is a diagram showing a state of the progress information display portion of a third example at the time of starting observation in the above first embodiment;

FIG. 8 is a diagram showing a state of the progress information display portion of the third example during the observation in the above first embodiment;

FIG. 9 is a diagram showing a state of the progress information display portion of a fourth example during observation in the above first embodiment;

FIG. 10 is a diagram showing a state of the progress information display portion of a fifth example during observation in the above first embodiment;

FIG. 11 is a block diagram showing a configuration related to a control portion of an endoscope apparatus in a second embodiment of the present invention;

FIG. 12 is a diagram showing an example of the progress information display portion during observation in the above second embodiment;

FIG. 13 is a block diagram showing a configuration related to the control portion of an endoscope apparatus in a third embodiment;

FIG. 14 is a diagram showing an example of an observed area and an unobserved area when calyces are being observed by an endoscope in the above third embodiment;

FIG. 15 is a diagram showing an example of progress information generated by a progress information generating portion in the observation state shown in FIG. 14, in the above third embodiment;

FIG. 16 is a diagram showing an example of the observed area and the unobserved area when the observation has progressed to some degree from the observation state shown in FIG. 14, in the above third embodiment;

FIG. 17 is a diagram showing an example of progress information generated by the progress information generating portion in the observation state shown in FIG. 16, in the above third embodiment;

FIG. 18 is a diagram showing an example at the time when the observation has been completed, and only the observed area exists in the above third embodiment;

FIG. 19 is a diagram showing an example of progress information generated by the progress information generating portion in the observation completion state shown in FIG. 18, in the above third embodiment; and

FIG. 20 is a diagram showing an example of displaying the progress information shown in FIG. 19 being superimposed on a three-dimensional model image, in the above third embodiment.

### DETAILED DESCRIPTION OF THE PREFERRED EMBODIMENTS

Embodiments of the present invention will be described below with reference to drawings.

### First Embodiment

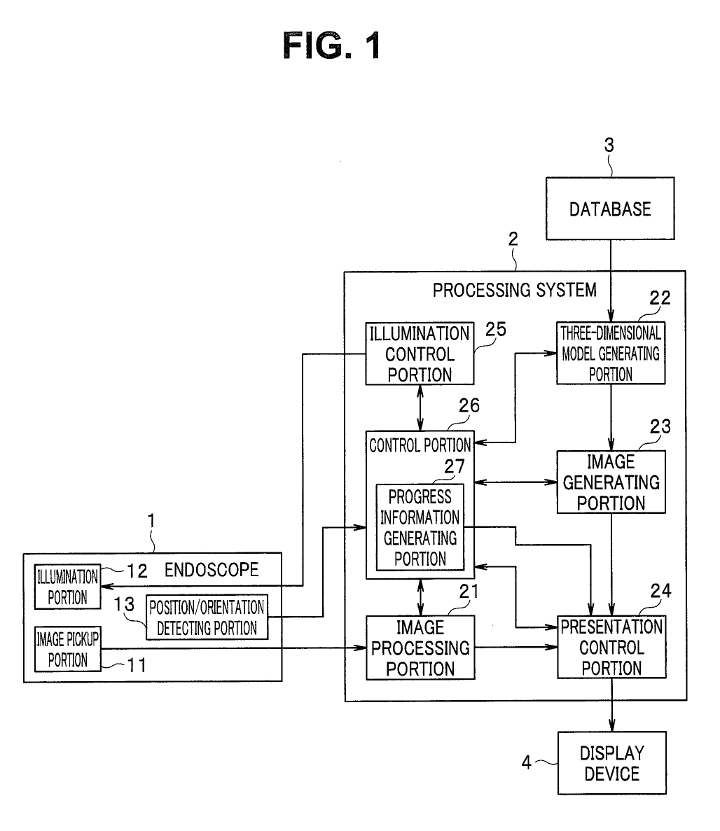

FIGS. 1 to 10 show a first embodiment of the present invention, and FIG. 1 is a block diagram showing a configuration of an endoscope apparatus.

The endoscope apparatus is provided with an endoscope 1, a processing system 2 and a display device 4 and may be further provided with a database 3 as necessary. Description will be made below on a case where the database 3 is not provided, as an example. As for a case where the database 3 is provided, the case will be appropriately described.

The endoscope 1 is an image acquisition apparatus which, in order to observe an inside of a subject having a three-dimensional shape, acquires an image of the inside of the subject and is provided with an image pickup portion 11, an illumination portion 12 and a position/orientation detecting portion 13. The image pickup portion 11, the illumination portion 12 and the position/orientation detecting portion 13 are, for example, arranged on a distal end portion of an insertion portion of the endoscope 1 which is to be inserted into a subject.

Note that though renal pelvis calyces of a kidney are given as an example of a subject having a three-dimensional shape in the present embodiment, the present embodiment is not limited to renal pelvis calyces but is widely applicable to any subject if the subject has a plurality of ducts and endoscopic observation can be performed for the subject.

The illumination portion 12 radiates illumination light to an inside of a subject.

The image pickup portion 11 forms, by an optical system, an optical image of the inside of the subject to which the illumination light is radiated and performs photoelectric conversion by an image pickup device and the like to generate a picked-up image signal.

The position/orientation detecting portion 13 detects a three-dimensional position of the distal end portion of the insertion portion of the endoscope 1 to output the three-dimensional position as position information, and detects a direction to which the distal end portion of the insertion portion of the endoscope 1 faces to output the direction as orientation information. For example, if an xyz coordinate system is set, the position information is indicated by (x, y, z) coordinates, and the orientation information is indicated by an angle around an x axis, an angle around a y axis and an angle around a z axis (therefore, the position/orientation detecting portion 13 is also called, for example, a 6D sensor). Note that the position information and the orientation information about the endoscope 1 may be indicated by using any other appropriate method (for example, a polar coordinate system).

The processing system 2 is such that performs control of the endoscope 1, communicates with the database 3 as necessary, processes a picked-up image signal, position information and orientation information acquired from the endoscope 1 to generate image data for display or image data for recording, and outputs the image data to the display device 4 and the like. Note that the processing system 2 may be configured as a single apparatus or may be configured with a plurality of apparatuses such as a light source apparatus and a video processor.

The processing system 2 is provided with an image processing portion 21, a three-dimensional model generating portion 22, an image generating portion 23, a presentation control portion 24, an illumination control portion 25 and a control portion 26.

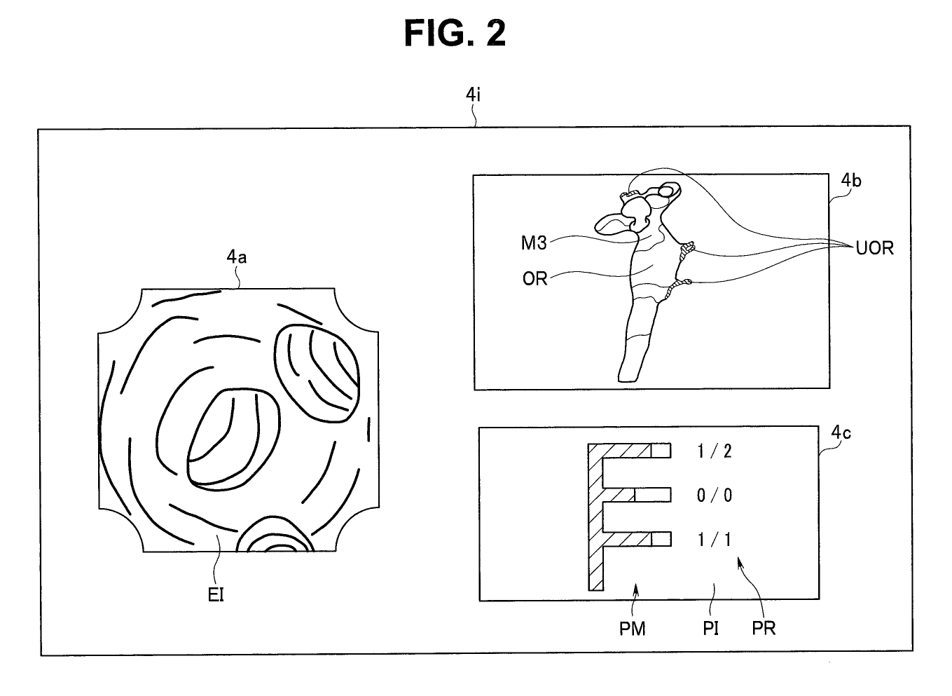

The image processing portion 21 generates a picked-up image from a picked-up image signal outputted from the image pickup portion 11 and performs various kinds of image processings, such as demosaicking processing (or synchronization processing), white balance processing, color matrix processing and gamma conversion processing, for the generated picked-up image to generates an endoscopic image EI (see FIG. 2).

The three-dimensional model generating portion 22 generates three-dimensional model data of a subject. For example, the three-dimensional model generating portion 22 acquires endoscopic images EI generated by the image processing portion 21 (or endoscopic images EI image-processed by the image processing portion 21 to generate a three-dimensional model) and position information and orientation information detected by the position/orientation detecting portion 13 when picked-up images from which the endoscopic images EI have been generated were picked up, corresponding to a plurality of frames via the control portion 26.

Then, the three-dimensional model generating portion 22 is adapted to generate stereoscopic three-dimensional model data while causing a position relationship among the endoscopic images EI of the plurality of frames to be adjusted based on the position information and the orientation information about each frame. In this case, three-dimensional model data is gradually constructed as observation progresses, and therefore generation of a three-dimensional model image M3 (see FIG. 2 and the like) by the image generating portion 23 gradually progresses.

The method of generating the three-dimensional model data by the three-dimensional model generating portion 22 is not limited to the above. For example, if the endoscopic examination for the subject is endoscopic examination for second or subsequent time, and three-dimensional model data generated in the past endoscopic examinations is already recorded in the database 3, the three-dimensional model data may be used. Or if data acquired by performing contrast enhanced CT imaging for the subject is already recorded in the database 3, three-dimensional model data may be generated using the contrast enhanced CT data.

In the database 3, a renal pelvis calyx model to be a basis of a progress map PM as shown in FIG. 2 to be described later is stored in advance. Here, the stored renal pelvis calyx model may be, for example, a standard renal pelvis calyx model (that is, a model based on an average renal pelvis calyx shape of a human body), renal pelvis calyx models of a plurality of patterns classified based on a lot of cases, which have recently been proposed, a renal pelvis calyx model generated by modeling three-dimensional model data of a subject or any other model (that is, the renal pelvis calyx model is not limited to a particular model). Further, the renal pelvis calyx model is not limited to being stored in the database 3 but may be stored in a storage device or the like that the control portion 26 in the processing system 2 is provided with.

The image generating portion 23 generates a three-dimensional image M3 (see FIG. 2 and the like) based on the three-dimensional model data generated by the three-dimensional model generating portion 22. The three-dimensional model image M3 is, for example, an image when a three-dimensional subject image is seen in a certain line-of-sight direction, and the line-of-sight direction is changeable (that is, the three-dimensional model image M3 rotates accompanying a change in the line-of-sight direction). Note that the three-dimensional model generating portion 22 and image generating portion 23 described above constitute a three-dimensional model image generating portion.

The presentation control portion 24 presents progress information PI (see FIG. 2 and the like) generated by a progress information generating portion 27 to be described later in association with the three-dimensional model image M3 generated by the image generating portion 23. Here, the presentation control portion 24 may associate the three-dimensional model image M3 and the progress information PI by presenting the three-dimensional model image M3 and the progress information PI side by side (see FIG. 2 and the like). Or the presentation control portion 24 may associate the three-dimensional model image M3 and the progress information PI by superimposing the progress information PI on the three-dimensional model image M3 to present the progress information PI and the three-dimensional model image M3. The presentation control portion 24 also presents the endoscopic images EI generated by the image processing portion 21. Since presentation of the progress information PI, the three-dimensional model image M3 and the endoscopic images EI by the presentation control portion 24 is output to the display device 4 or a recording device not shown (the recording device may be the database 3), the presentation control portion 24 can be also called an output information control portion.

The illumination control portion 25 is such that controls on/off or an amount of illumination light radiated by the illumination portion 12. Here, the illumination control portion 25 and the illumination portion 12 may be a light source device and a light guide or the like, respectively. Or the illumination control portion 25 and the illumination portion 12 may be a light emission control circuit and a light emission source such as an LED, respectively.

The control portion 26 is such that controls the whole processing system 2 and further controls the endoscope 1. The control portion 26 is connected to the image processing portion 21, the three-dimensional model generating portion 22, the image generating portion 23, the presentation control portion 24 and the illumination control portion 25 which have been described above.

The control portion 26 is provided with the progress information generating portion 27 configured to generate progress information PI showing a progress state of observation of a subject by the endoscope 1. A specific example of the progress information PI generated by the progress information generating portion 27 will be described later with reference to drawings.

The database 3 is connected to the processing system 2, for example, via an in-hospital system, and three-dimensional model data of subjects generated based on contrast enhanced CT data of the subjects, three-dimensional model data of the subjects generated based on the contrast enhanced CT data, three-dimensional model data of the subjects generated by past endoscopic examinations, or a renal pelvis calyx model to be a basis of a progress map PM are recorded.

The display device 4 is configured including one or more monitors and the like and displays a presentation image including an endoscopic image EI, a three-dimensional model image M3 and progress information PI outputted from the presentation control portion 24.

FIG. 2 is a diagram showing a state of a display screen 4i of the display device 4 including a progress information display portion 4c of a first example, during observation.

On the display screen 4i, an endoscopic image display portion 4a, a three-dimensional model image display portion 4b and a progress information display portion 4c are provided.

On the endoscopic image display portion 4a, an endoscopic image EI generated by the image processing portion 21 is displayed.

On the three-dimensional model image display portion 4b, a three-dimensional model image M3 generated by the image generating portion 23 is displayed. Since the three-dimensional model image M3 shown in FIG. 2 is such a three-dimensional model image M3 constructed as observation progresses, as described above, an observed area OR which has already been observed is displayed, and it is displayed that unobserved areas UOR exist, by causing a display aspect (for example, a color (hue, saturation, brightness), a pattern, a combination of color and pattern, or the like) of connection parts to the unobserved areas UOR to be different. Some specific examples are: displaying the unobserved areas UOR with a red hue (red display), displaying the unobserved areas UOR with a lower saturation (monochrome display), displaying the unobserved areas with a higher brightness (highlight display) and the like. An aspect of causing the unobserved areas UOR displayed here to be blinkingly displayed in order to further enhancingly display the unobserved areas UOR is also possible.

On the progress information display portion 4c, progress information PI is displayed. Note that though the progress information display portion 4c is a display portion that is a little smaller than the three-dimensional model image display portion 4b in the shown example, the display position and display size of each display portion may be changeable as described later.

The progress information PI includes, for example, a progress map PM and a calculus mark display PR.

The progress map PM is such that, for an observation target (here, for example, a kidney), the renal pelvis calyx structure of the observation target is modeled and displayed, and display aspects (for example, colors, patterns, combinations of color and pattern, or the like as described above) of observed areas OR and unobserved areas UOR are caused to be different (in FIG. 2, it is indicated by hatching that the display aspects are different).

More specifically, a kidney is provided with calyces which are a plurality of partial areas forming a duct structure. Therefore, for example, information showing a ratio of the number of observed calyces to the total number of calyces of the kidney (or the total number of calyces estimated to be included in the kidney) can be displayed by causing the display aspects to be different.

More particularly, the calyces are classified into superior calyces, middle calyces and inferior calyces; and when progress information PI for each of the parts is displayed, a ratio of the number of observed calyces among the superior calyces to the total number of calyces existing as the superior calyces is displayed on the part for the superior calyces in the progress map PM, and results calculated similarly can be displayed for the middle calyces and the inferior calyces, respectively (see FIG. 2 and the like).

Thus, it is possible to, by seeing the progress map PM, intuitively and more easily determine what percentage of the total number of observation targets has been observed.

The progress information PI, however, is not limited to being calculated based on the ratio of the number of partial areas but may be calculated based on a ratio of volume or a ratio of area.

In the case of performing calculation based on a ratio of volume, a ratio of volume of observed areas OR to volume of a prespecified area of a subject, for example, volume of all areas of the subject (if it is not known, estimated volume of all the areas of the subject) can be calculated and used as progress information PI.

In the case of performing calculation based on a ratio of area, a ratio of area of the observed area OR to area of the prespecified area of the subject, for example, area of all areas of the subject (if it is not known, estimated area of all the areas of the subject) can be calculated and used as progress information PI.

Or instead of calculating a ratio as progress information PI, the total number of partial areas the subject is provided with and the number of observed partial areas may be used as progress information PI.

In addition, the number of unobserved partial areas may be displayed as progress information PI (together with the total number of partial areas as necessary). Here, the number of unobserved partial areas is calculated by subtracting the number of observed partial areas from an estimated total number of partial areas.

Note that judgment that a calyx has been observed is not limited to being made by observation of an inside of the calyx having completely been (that is, 100%) performed. For example, the judgment may be made by 80% of the observation of the inside of the calyx having been performed, or an arbitrary ratio may be set beforehand.

Though the progress map PM shown in FIG. 2 adopts a standard model in which calyces are separated into superior calyces, middle calyces and inferior calyces, the progress map PM is not limited to the above, and a more detailed model may be used. For example, if there are a plurality of renal pelvis calyx models classified based on a lot of cases as described above, and three-dimensional model data of a subject already exists, an appropriate model may be selected from among the plurality of renal pelvis calyx models based on the three-dimensional model data and used as a progress map PM. A progress map PM generated by modeling the three-dimensional model data of the subject may be used as described above. Or a three-dimensional model image of a subject may be used as a progress map PM as described later.

The calculus mark display PR is a part where information showing the number of already marked targets relative to the number of targets to be marked is displayed. The targets to be marked in the present embodiment are, for example, calculi. That is, the number of calculi which have already been marked is displayed relative to the number of calculi acquired in advance by another method (for example, simple CT imaging).

More specifically, in the example shown in FIG. 2, a state is shown in which one of two calculi existing in the superior calyces has already been marked, no calculus exists in the middle calyces, and one calculus existing in the inferior calyces has already been marked.

Note that in the example shown in FIG. 2, the display positions and display sizes of the endoscopic image display portion 4a, the three-dimensional model image display portion 4b and the progress information display portion 4c may be adapted to be independently changed as desired. As an example, the endoscopic image display portion 4a is displayed large on a right side of the display screen 4i; the progress information display portion 4c is displayed small on an upper left of the display screen 4i; and the three-dimensional model image display portion 4b is displayed in a moderate size on a lower left. For example, if each of the endoscopic image display portion 4a, the three-dimensional model image display portion 4b and the progress information display portion 4c is displayed as one window, it is possible to easily perform the change in the display positions and display sizes as described above.

Though one display screen 4i is provided in the example shown in FIG. 2 on an assumption that the display device 4 is configured with one monitor, display may be separately performed on a plurality of monitors as described above. For example, the display device 4 may be configured being provided with two monitors so that the endoscopic image display portion 4a is displayed on a first monitor, and the three-dimensional model image display portion 4b and the progress information display portion 4c are displayed on a second monitor. Furthermore, the display device 4 may be configured being provided with three monitors so that the endoscopic image display portion 4a, the three-dimensional model image display portion 4b and the progress information display portion 4c are displayed on the different monitors, respectively.

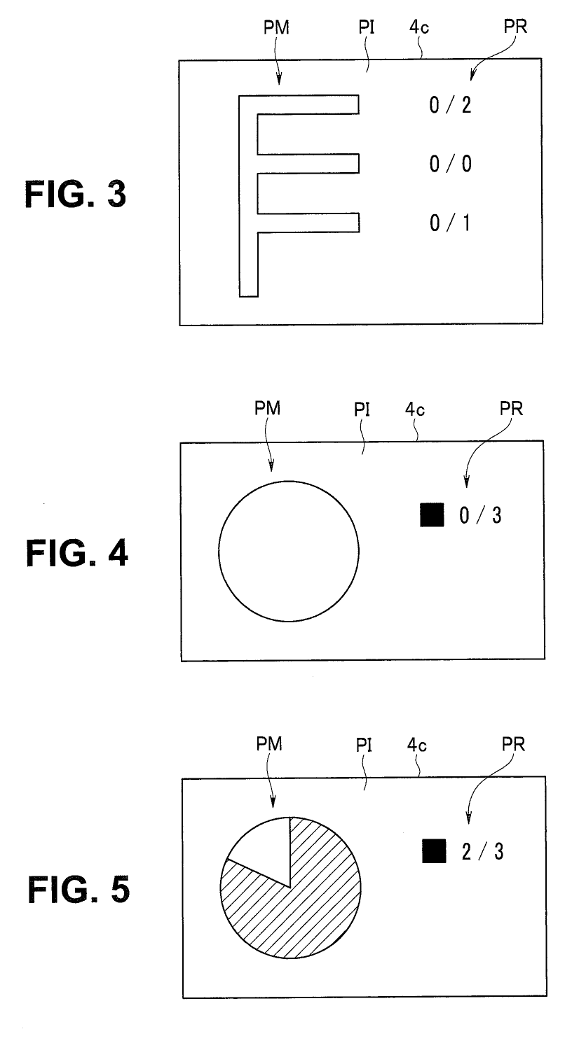

FIG. 3 is a diagram showing a state of the progress information display portion 4c of the first example at the time of starting observation.

As shown in FIG. 3, when observation is started, the whole progress map PM is in a display aspect corresponding to unobserved areas UOR, and the calculus mark display PR shows that the number of marked calculi is 0.

FIG. 4 is a diagram showing a state of the progress information display portion 4c of a second example at the time of starting observation, and FIG. 5 is a diagram showing a state of the progress information display portion 4c of the second example during the observation.

In the second example of the progress information display portion 4c shown in FIGS. 4 and 5, a pie graph is merely displayed as a progress map PM. Since the progress map PM is not classified into superior calyces, middle calyces and inferior calyces, the calculus mark display PR is such that displays how many calculi have been marked relative to three calculi existing in all the calyces of the kidney.

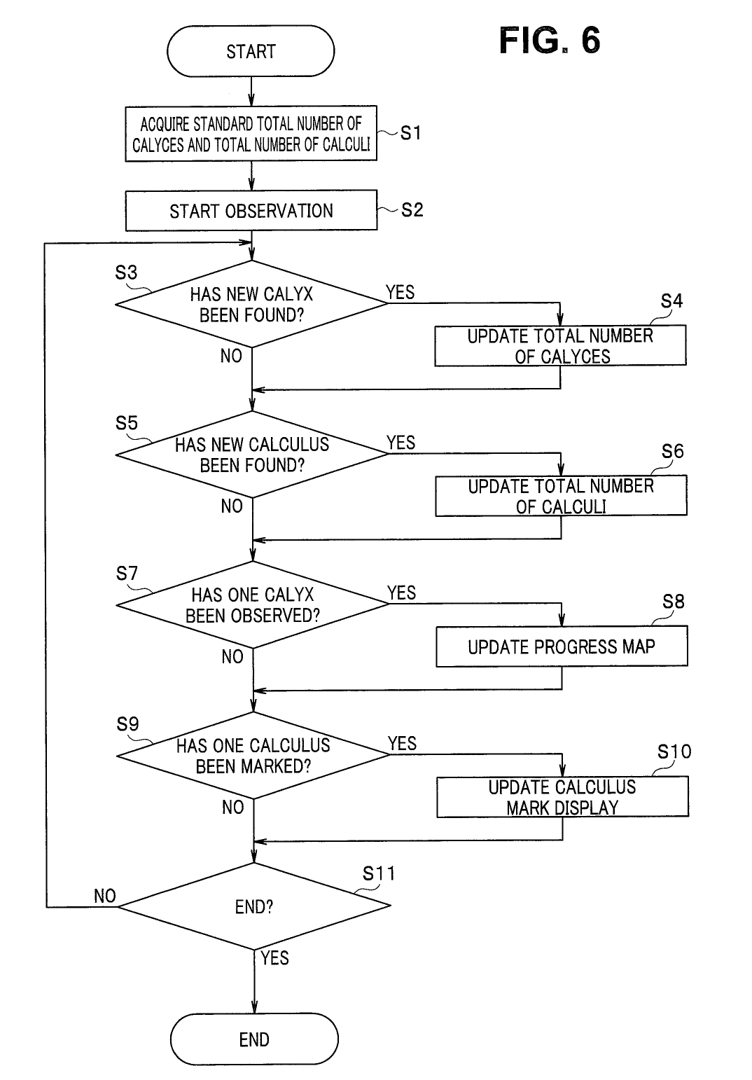

FIG. 6 is a flowchart showing operation of the endoscope apparatus. Note that here, since an accurate shape of renal pelvis calyces of a subject is not known yet, an example of displaying progress information PI based on a standard renal pelvis calyx model will be described.

When the process is started, the total number of calyces based on the standard renal pelvis calyx model is acquired, and the total number of calculi of the subject which is already known is acquired first (step S1). Here, as for the number of calculi of the subject, it is desirable to acquire how many calculi exist, for example, in superior calyces, middle calyces and inferior calyces, respectively, but how many calculi exist in all the calyces may also be acquired as shown in FIGS. 4 and 5.

Then, observation of the calyces by the endoscope 1 is started (step S2).

During the observation of the calyces, it is judged whether a new calyx different from the standard renal pelvis calyx model has been found or not (step S3). If a new calyx is found, the total number of calyces to be observed is updated (step S4).

If the process of step S4 is performed, or if it is judged at step S2 that a new calyx has not been found, it is judged whether a new calculus other than the calculi acquired at step S1 has been found or not (step S5). If a new calculus has been found, the total number of calculi is updated (step S6).

If the process of step S6 is performed, or if it is judged at step S5 that a new calculus has not been found, it is judged whether one calyx has been observed or not (step S7).

Here, if it is judged that one calyx has been observed, a progress map PM showing a ratio of the number of observed calyces to the total number of calyces is generated, and display of the progress information display portion 4c is updated with the generated progress map PM (step S8). At this time, as shown in FIGS. 2 and 3, it is preferable to generate a progress map PM showing what percentage of observation has been performed for superior calyces, middle calyces and inferior calyces, respectively, because observation can be more efficiently progressed.

If the process of step S8 is performed, or it is judged at step S7 that a calyx has not been observed, it is judged whether one calculus has newly been marked or not while the flow proceeds along the loop of step S3 described above to step S11 to be described later (step S9). If one calculus has been marked, the calculus mark display PR is updated (step S10).

After that, it is judged whether or not to end the endoscopic observation (step S11). If the endoscopic observation is not to be ended, the flow returns to step S3 described above, and the endoscopic observation is continued.

On the other hand, if it is judged at step S11 that the endoscopic observation is to be ended, the process is ended.

Note that though it is assumed in the above description that the accurate shape of the renal pelvis calyces of the subject is unknown at the stage of starting the endoscopic observation, it is possible to, in a case where the shape of the renal pelvis calyces is known beforehand, such as a case where the endoscopic observation is endoscopic observation for second or subsequent time or a case where contrast enhanced CT data is acquired beforehand, display the progress information PI more appropriately by using a renal pelvis calyx model adapted for the subject.

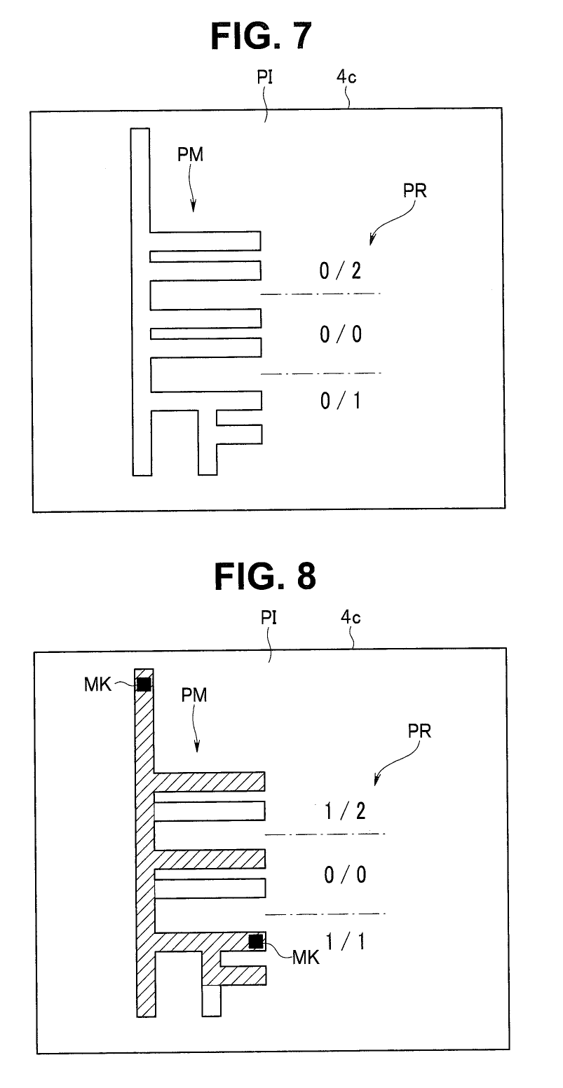

An example of using a renal pelvis calyx model adapted for a subject will be described with reference to FIGS. 7 and 8. FIG. 7 is a diagram showing a state of the progress information display portion 4c of a third example at the time of starting observation, and FIG. 8 is a diagram showing a state of the progress information display portion 4c of the third example during the observation.

In the third example shown in FIGS. 7 and 8, a progress map PM displayed in the progress information display portion 4c is based on a more detailed renal pelvis calyx model adapted for a shape of renal pelvis calyces of a subject. Furthermore, if a calculus is marked, a mark MK showing that the marked calculus exists is displayed at a position almost corresponding to a position of the marked calculus on the progress map PM (that is, the progress information generating portion 27 generates progress information such that the mark MK is included) as shown in FIG. 8.

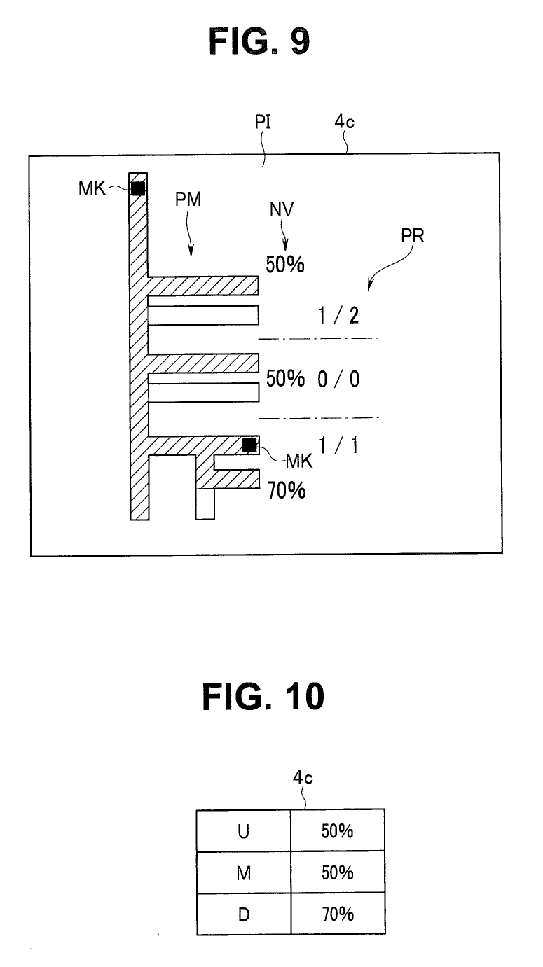

FIG. 9 is a diagram showing a state of the progress information display portion 4c of a fourth example during observation.

If a shape of renal pelvis calyces of a subject is unknown, a standard renal pelvis calyx model is used as a progress map PM, and progress information display is display showing an approximate degree of progress. If the shape of the renal pelvis calyces of the subject is known before endoscopic observation, a ratio of volume (or area) of observed areas OR to volume (or area) of all areas of the subject is information showing a degree of progress with a high accuracy as described above. In this case, progress rates NV may be further displayed as progress information PI as shown in FIG. 9. In the example in FIG. 9, the progress rates NV are displayed with percent values, and it is shown that observation of superior calyces, observation of middle calyces, and observation of inferior calyces have been completed 50%, 50% and 70%, respectively.

Note that though display by percentage is performed for the superior calyces, the middle calyces and the inferior calyces, respectively, here, display by percentage may be performed for each of all calyces or only for calyces in which calculi exist, in more detail.

FIG. 10 is a diagram showing a state of the progress information display portion 4c of a fifth example during observation.

In the example shown in FIG. 10, display of the progress information display portion 4c is further simplified, and that observation of superior calyces (U), observation of middle calyces (M), and observation of inferior calyces (D) have been completed 50%, 50% and 70%, respectively, is shown, for example, as numerical values in a table. In this case, it is, of course, possible to further add and display the number of calculi for which calculus mark display has been completed relative to the total number of calculi, similarly to each of the examples described above.

According to the first embodiment as described above, since progress information PI showing a progress state of observation of a subject by the endoscope 1 is generated and presented in association with a three-dimensional model image M3, it is possible to intuitively and more easily grasp a progress state of endoscopic observation, that is, a state about which stage the endoscopic observation has progressed to, so that usability is improved.

Further, since the progress information PI is adapted to include information showing a ratio of volume of observed areas OR to volume of all areas of the subject, accurate progress state display based on a volume ratio becomes possible.

Or if the progress information PI is adapted to include information showing a ratio of area of the observed area OR to area of all the areas of the subject, accurate progress state display based on an area ratio becomes possible.

If the progress information PI is adapted to include information showing a ratio of the number of observed partial areas to the total number of partial areas that the subject is provided with, it becomes possible to grasp a remaining process of the endoscopic observation in units of the number of partial areas.

In addition, since the progress information PI is adapted to further include information showing the number of targets (here, calculi) which have already been marked relative to the number of targets (calculi) to be marked, it becomes possible to easily grasp which stage marking of targets has progressed to.

Since the progress information PI and the three-dimensional model image M3 are presented side by side, it is possible to grasp more appropriately, for a three-dimensional observation target, up to which part endoscopic observation has been performed. Thereby, it is possible to prevent oversight of an unobserved area UOR existing at a position that cannot be visually confirmed.

Furthermore, even if an unobserved area UOR is hidden on a back side of the three-dimensional model image M3 when the use see the three-dimensional model image M3, the user can confirm existence of the unobserved area UOR by the progress information PI. Thereby, it is also possible to prevent oversight of an unobserved area UOR existing at a position that cannot be visually confirmed.

### Second Embodiment

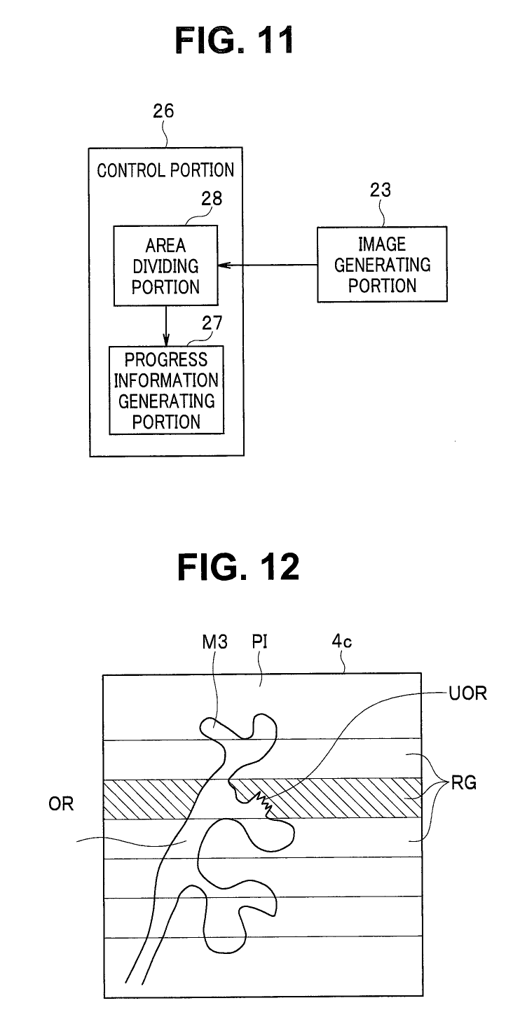

FIGS. 11 and 12 show a second embodiment of the present invention; FIG. 11 is a block diagram showing a configuration related to the control portion 26 of an endoscope apparatus; and FIG. 12 is a diagram showing a state of the progress information display portion 4c during observation.

In the second embodiment, parts similar to parts of the first embodiment described above are given the same reference numerals, and description will be appropriately omitted. Description will be made mainly only on different points.

As shown in FIG. 11, the control portion 26 of the present embodiment is further provided with an area dividing portion 28 in addition to the progress information generating portion 27.

The area dividing portion 28 divides a three-dimensional model image M3 generated by the image generating portion 23 into a plurality of divided areas RG (see FIG. 12) together with a background image.

The progress information generating portion 27 performs image processing of at least one of the three-dimensional model image and the background image in a divided area RG including an unobserved area UOR, among the plurality of divided areas RG divided by the area dividing portion 28, so that the image is distinguishable from the other divided areas RG not including an unobserved area UOR in order to generate progress information PI. Since the progress information generating portion 27 generates information for grasping a progress state of endoscopic observation in a bird's eye view, the progress information generating portion 27 can be also called a bird's eye view information generating portion.

In the present embodiment, the three-dimensional model image M3 and the image-processed background image described above are used as a progress map PM as shown in FIG. 12. In this case, a three-dimensional model image M3 similar to the three-dimensional model image M3 of the three-dimensional model image display portion 4b may be displayed on the progress information display portion 4c, or the three-dimensional model image display portion 4b may also serve as the progress information display portion 4c. That is, the progress information PI is not limited to be displayed on the progress information display portion 4c provided separately from the three-dimensional model image display portion 4b but may be displayed being superimposed on the three-dimensional model image M3 of the three-dimensional model image display portion 4b.

Note that since the three-dimensional model image M3 of the three-dimensional model image display portion 4b is, for example, rotatable as described above, such a configuration is also possible that, in the case of displaying a three-dimensional model image M3 similar to the three-dimensional model image of the three-dimensional model image display portion 4b on the progress information display portion 4c, the three-dimensional model image M3 of the progress information display portion 4c also rotates in synchronization with rotation of the three-dimensional model image M3 of the three-dimensional model image display portion 4b.

In the example shown in FIG. 12, the three-dimensional model image M3 and the background image are divided in the plurality of divided areas RG (here, a plurality of divided areas RG each of which forms a band shape in a horizontal direction). In this case, a display aspect of the background image corresponding to the divided area RG including the unobserved area UOR is caused to be different from a display aspect of the background image corresponding to the other divided areas RG so that the divided area RG that includes the unobserved area UOR is distinguishable.

Here, instead of causing the display aspect of the background image to be different, the display aspect of the three-dimensional model image M3 may be caused to be different, or the display aspects of the background image and the three-dimensional model image M3 may be caused to be different.

The example shown in FIG. 12 shows a case where there is one divided area RG that includes an unobserved area UOR. In a case where there are a plurality of divided areas RG each of which includes an unobserved area UOR, however, there are also a plurality of parts the display aspects of which are caused to be different as described above.

In this case, the display aspects may be caused to be gradually different according to sizes and the like of the unobserved areas UOR. That is, for a divided area RG including a small unobserved area UOR, the display aspect may be caused to be different a little. For a divided area RG including a large unobserved area UOR, the display aspect may be caused to be significantly different. For example, a divided area RG including a small unobserved area UOR may be displayed being painted in light color, and a divided area RG including a large unobserved area UOR may be displayed being painted in deep color.

Note that in the case of adopting such a three-dimensional model image M3 that is constructed as endoscopic observation progresses as described above, only a constructed part may be divided into divided areas RG.

According to the second embodiment as described above, advantageous effects almost similar to the advantageous effects of the first embodiment described above are obtained; and since progress information PI is presented being superimposed on a three-dimensional model image M3, it is not necessary to compare the three-dimensional model image M3 and the progress information PI and it is possible to grasp a progress state of endoscopic observation only by seeing the three-dimensional model image M3.

Since a display aspect showing whether an unobserved area UOR is included or not is caused to be different for each divided area RG, it is possible to grasp a gradual progress state for each area.

### Third Embodiment

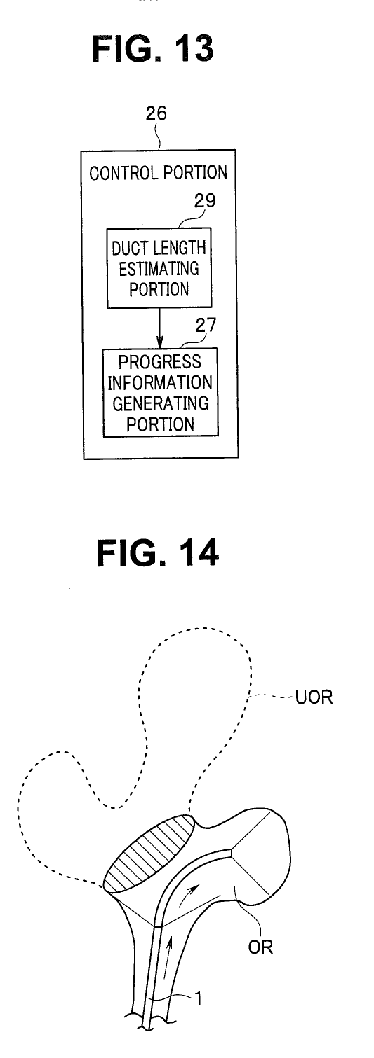

FIGS. 13 to 20 show a third embodiment of the present invention; and FIG. 13 is a block diagram showing a configuration related to the control portion 26 of an endoscope apparatus.

In the third embodiment, parts similar to the first and second embodiments described above are given the same reference numerals, and description will be appropriately omitted. Description will be made mainly only on different points.

As shown in FIG. 13, the control portion 26 of the present embodiment is further provided with a duct length estimating portion 29 in addition to the progress information generating portion 27.

The duct length estimating portion 29 detects lengths of one or more observed ducts among a plurality of ducts that a subject includes, and estimates a length of an unobserved duct based on the detected lengths of the observed ducts.

The progress information generating portion 27 generates core line information about the observed ducts, generates core line information about the unobserved duct based on the lengths of the unobserved ducts estimated by the duct length estimating portion 29, and generates progress information PI in which the core line information about the observed ducts and the core line information about the unobserved duct are displayed in display aspects enabling both of the pieces of core line information to be distinguishable from each other. The progress information PI generated by the progress information generating portion 27 is displayed on the progress information display portion 4c as a progress map PM.

More specifically, it is assumed that calyces as ducts are observed by the endoscope 1, and one calyx becomes an observed area OR as shown in FIG. 14. Here, FIG. 14 is a diagram showing an example of an observed area OR and an unobserved area UOR when calyces are being observed by the endoscope 1.

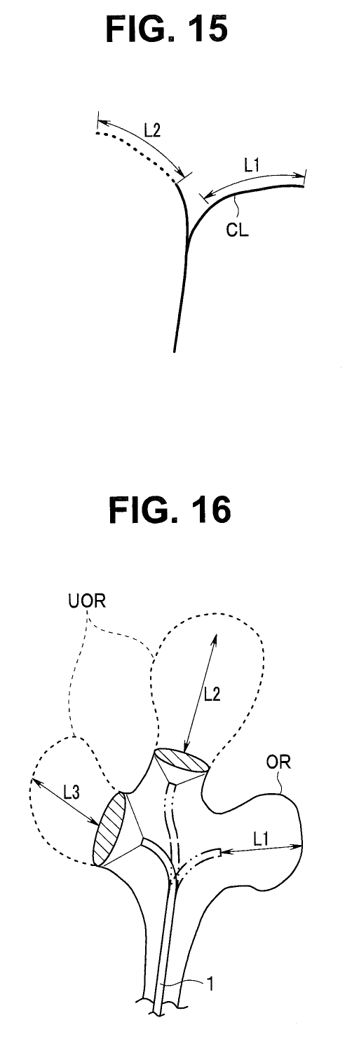

In this case, the duct length estimating portion 29 detects a length L1 of a duct of the observed area OR as shown in FIG. 15, for example, based on three-dimensional model data generated by the three-dimensional model generating portion 22. FIG. 15 is a diagram showing an example of the progress information PI generated by the progress information generating portion 27 in the observation state shown in FIG. 14.

If the observed area OR is such a range as indicated by a solid line in FIG. 14, it is not known yet that there are two calyces in the unobserved area UOR as indicated by a dotted line in FIG. 14. Therefore, the duct length estimating portion 29 estimates that there is one unobserved calyx. Then, the duct length estimating portion 29 estimates a length L2 of the one calyx in the unobserved area UOR, which is an unobserved duct, based on the detected length L1 of the duct of the observed area OR.

The estimation is performed on an assumption of L2=L1, for example, based on an assumption that sizes (or depths) of respective calyces are almost the same. If there are a plurality of observed calyces, and lengths of ducts of the plurality of calyces are already detected, an average value of the detected lengths, for example, can be set as the estimated length of the unobserved calyx.

Then, the progress information generating portion 27 generates core line information CL about the calyx in the observed area OR as indicated by a solid line in FIG. 15, based on the three-dimensional model data generated by the three-dimensional model generating portion 22 (or, in addition, the length L1 of calyx in the observed area OR detected by the duct length estimating portion 29).

Furthermore, based on the length L2 of the calyx in the unobserved area UOR estimated by the duct length estimating portion 29, the progress information generating portion 27 generates core line information as indicated by a dotted line in FIG. 15 by extrapolating a curved line of the core line of the observed area OR to extend the curved line by the length L2. Thereby, it is possible to generate core line information about the whole observation target including the observed area OR and the unobserved area UOR (core line information showing a virtual overall shape of the observation target) even if the endoscopic observation is not endoscopic observation for second or subsequent time, or even if there is no contrast enhanced CT data.

At this time, the progress information generating portion 27 generates progress information PI by causing display aspects (for example, colors, patterns, or combinations of color and pattern as described above) of the core line of the observed area OR and the core line of the unobserved area UOR to be different so that the core line of the observed area OR and the core line of the unobserved area UOR are distinguishable from each other. As an example, one of the core lines of the observed area OR and the unobserved area UOR is shown as a red line, and the other is shown as a blue line. An aspect of causing the core line of the unobserved area UOR to be blinkingly displayed in order to further enhancing the unobserved area UOR displayed here is also possible.

By seeing the progress information PI as in FIG. 15 that is displayed on the progress map PM of the progress information display portion 4c, the user can grasp that at least one unobserved calyx remains.

It is assumed that the observation of the calyces by the endoscope 1 has progressed to a state as shown in FIG. 16 from the state as shown in FIG. 14. FIG. 16 is a diagram showing an example of the observed area OR and the unobserved area UOR when the observation has progressed to some degree from the observation state shown in FIG. 14.

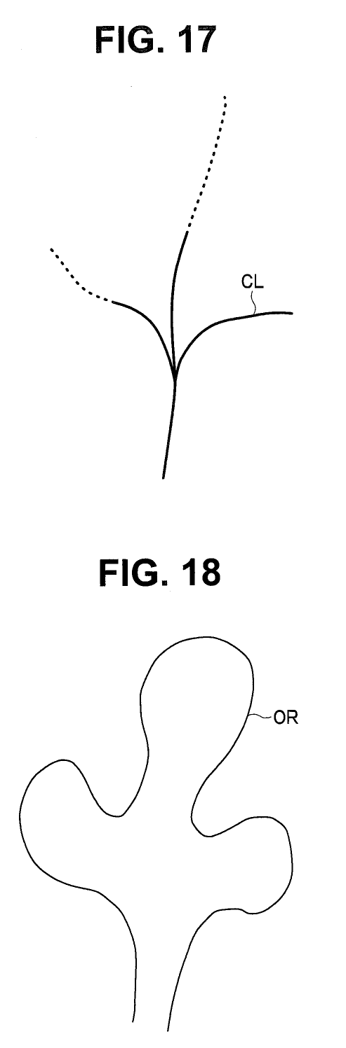

At this time, the duct length estimating portion 29 can estimate that there are two calyces in the unobserved area UOR. Therefore, the duct length estimating portion 29 estimates, for lengths L2 and L3 of the two calyces in the unobserved area UOR, which are unobserved ducts, that L2=L1 and L3=L1 are satisfied, based on the detected length L1 of the duct of the observed area OR. Thereby, the progress information generating portion 27 generates the core line information CL as indicated by solid lines and dotted lines in FIG. 17. Here, FIG. 17 is a diagram showing an example of the progress information PI generated by the progress information generating portion 27 in the observation state shown in FIG. 16. Thus, in the observation state shown in FIG. 16, two pieces of core line information CL are generated for the unobserved area UOR.

By seeing the progress information PI as in FIG. 17 that is displayed on the progress map PM of the progress information display portion 4c, the user can determine that two unobserved calyces remain.

It is assumed that the observation of the calyces by the endoscope 1 has further progressed to a state as shown in FIG. 18 from the state as shown in FIG. 16. Here, FIG. 18 is a diagram showing an example at the time when the observation has been completed, and only the observed area OR exists.

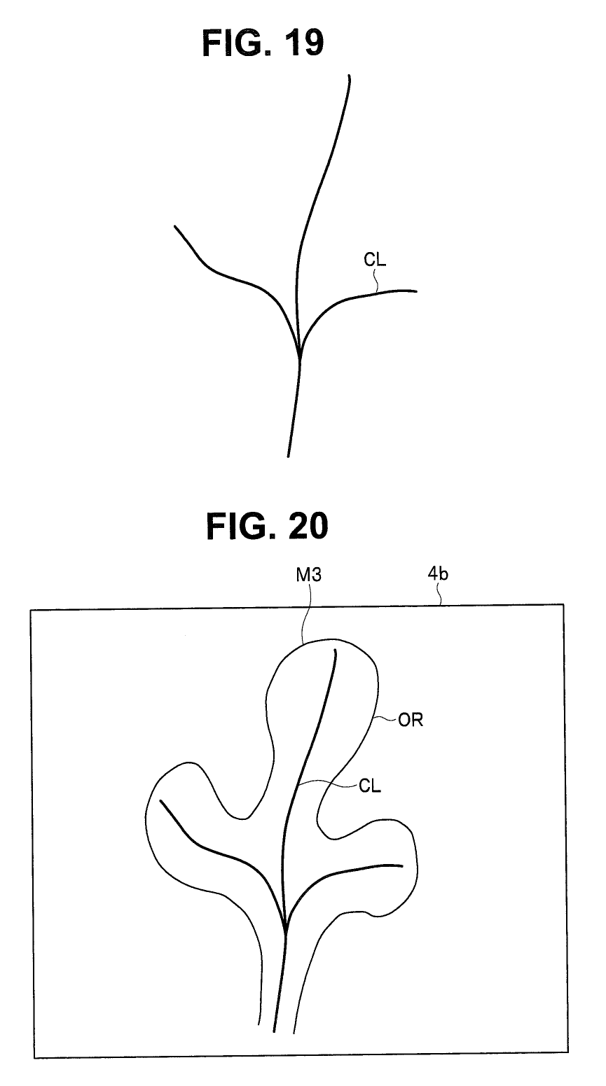

At this time, based on the core line information about the observed area OR detected by the duct length estimating portion 29, the progress information generating portion 27 generates core line information CL as indicated by solid lines in FIG. 19, that is, core line information CL in a display aspect indicating that all has been observed. FIG. 19 is a diagram showing an example of the progress information PI generated by the progress information generating portion 27 in the observation completion state shown in FIG. 18.

By seeing the progress information PI as in FIG. 19 that is displayed on the progress map PM of the progress information display portion 4c, the user can determine that the observation of the calyces has ended.

Note that since it is assumed in the above description that core line information CL is generated based on three-dimensional model data constructed as endoscopic observation progresses, only one core line that indicates being unobserved is displayed in the state shown in FIG. 15 though there are two unobserved calyces. In the case of generating core line information CL based on three-dimensional model data in which a renal pelvis calyx shape of a subject is already known (such as a case where endoscopic observation is second or subsequent endoscopic observation and a case where the three-dimensional model data is based on contrast enhanced CT data), however, core line shapes are specified in advance, and it is only necessary to cause display aspects to be different depending on whether observed or unobserved. Therefore, it is possible to grasp a degree of progress more accurately.

FIG. 20 is a diagram showing an example of displaying the progress information PI shown in FIG. 19 being superimposed on a three-dimensional model image M3.

Though the core line information CL generated by the progress information generating portion 27 may be displayed as a progress map PM of the progress information display portion 4c (that is, together with a three-dimensional model image M3 of the three-dimensional model image display portion 4b side by side), the core line information CL may be displayed being superimposed on the three-dimensional model image M3 of the three-dimensional model image display portion 4b as shown in FIG. 20. In this case, the three-dimensional model image display portion 4b also serves as the progress information display portion 4c.

By seeing the display as shown in FIG. 20, the user can easily determine to what extent observation of calyces displayed as a three-dimensional model image M3 has progressed.

According to the third embodiment as described above, advantageous effects almost similar to the advantageous effects of the first and second embodiments described above are obtained; and since a length of an unobserved duct is estimated based on a detected length of an observed duct to generate core line information about the observed and unobserved ducts, and such progress information PI that displays whether observed or unobserved in display aspects enabling whether observed or unobserved to be distinguishable, it is possible to easily recognize a degree of progress of endoscopic observation.

Note that it is also possible to configure the endoscope apparatus such that any of the display aspect of the first embodiment, the display aspect of the second embodiment and the display aspect of the third embodiment as described above can be adopted so that, in one endoscopic examination, the user can select and switch to a desired display aspect. In this case, the user makes a setting for switching to the desired display aspect, for example, by operating an operation portion provided on the endoscope 1, which is not shown, or an operation portion provided on the processing system 2, which is not shown.

Each portion described above may be configured as a circuit. An arbitrary circuit may be implemented as a single circuit or as a combination of a plurality of circuits as long as the same function can be achieved. Furthermore, the arbitrary circuit is not limited to being configured as a dedicated circuit for achieving an intended function, but a configuration is also possible in which the intended function is achieved by causing a general-purpose circuit to execute a processing program.

Though description has been made above mainly on an endoscope apparatus, the present invention may include an operation method for causing an endoscope apparatus to operate as described above, a processing program for causing a computer to perform a process similar to a process of the endoscope apparatus, a computer-readable non-transitory recording medium in which the processing program is recorded, and the like.

Note that the present invention is not limited to the above embodiments as they are, but the components can be modified and embodied within a range not departing from the spirit of the invention at a stage of practicing the invention. Further, various aspects of the invention can be formed by appropriately combining a plurality of components disclosed in the above embodiments. For example, some components may be deleted from all the components shown in an embodiment. Furthermore, components from different embodiments may be appropriately combined. Thus, various modifications and applications are, of course, possible within a range not departing from the spirit of the invention.

What is claimed is:

1. An endoscope apparatus comprising:
an endoscope configured to acquire an image of an inside of a subject; and
a processor including hardware; wherein
the processor generates three-dimensional model data of the subject;
generates a three-dimensional model image visually confirmable in a predetermined line-of-sight direction, based on the generated three-dimensional model data;
generates progress information enabling a progress state of observation by the endoscope to be visually confirmed as a ratio on an observation target based on the three-dimensional model data; and
associates the progress information with the three-dimensional model image and presents the progress information relative to the three-dimensional model image side by side.

2. The endoscope apparatus according to claim 1, wherein the progress information includes information showing a ratio of volume of an observed area to volume of a prespecified area of the subject.

3. The endoscope apparatus according to claim 1, further comprising a position/orientation detection sensor configured to detect position information and orientation information when the endoscope acquires the image; wherein
the processor generates the three-dimensional model data while the processor causes a position relationship among endoscopic images of a plurality of frames acquired by the endoscope to be adjusted based on the position information and the orientation information about each frame.

4. The endoscope apparatus according to claim 1, wherein
the subject includes a plurality of partial areas; and
the progress information includes information showing a ratio of a number of observed partial areas to a total number of partial areas that the subject includes.

5. The endoscope apparatus according to claim 1, wherein
the processor divides the three-dimensional model image into a plurality of divided areas with a background image; and
performs image processing of at least one of the three-dimensional model image and the background image of a divided area including an unobserved area, among the plurality of divided areas that have been divided, so that the image is distinguishable from the other divided areas not including the unobserved area to generate the progress information.

6. The endoscope apparatus according to claim 1, wherein
the processor detects lengths of one or more observed ducts among a plurality of ducts that the subject includes, and estimates a length of an unobserved duct based on the detected lengths of the observed ducts; and
generates core line information about the observed ducts, generates core line information about the unobserved duct based on the length of the unobserved duct estimated, and generates the progress information in which the core line information about the observed ducts and the core line information about the unobserved duct are displayed in such display aspects that both pieces of the core line information are distinguishable from each other.

7. The endoscope apparatus according to claim 1, wherein the progress information further includes information showing a number of already marked targets relative to a number of targets to be marked.

8. The endoscope apparatus according to claim 3, wherein the processor generates the three-dimensional model image where an observed area and an unobserved area are distinguishable from each other.
